(19) United States
(12) Patent Application Publication
(10) Pub. No.: US 2010/0272421 A1
(43) Pub. Date: Oct. 28, 2010

(54) ODOR GENERATING DEVICE

(75) Inventors: Chen-Chen Liu, Taipei (TW); Jiun-Ting Luo, Taipei (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: INVENTEC APPLIANCES CORP., Taipei (TW)

(21) Appl. No.: 12/765,999

(22) Filed: Apr. 23, 2010

(30) Foreign Application Priority Data
Apr. 23, 2009 (TW) 098113477

(51) Int. Cl. A61L 9/015

(52) U.S. Cl. 392/403

(57) ABSTRACT

The invention discloses an odor generating device, which is capable of generating an odor according to an atmosphere requirement. The odor generating device comprises a conductive portion and a controlling module. The controlling module is electrically connected to the conductive portion. The conductive portion has a capillary structure for accommodating a volatile liquid having the odor. The conductive portion comprises an electrode which is electrically connected to the controlling module. The controlling module apply a voltage, according to an atmosphere requirement, on the electrode of the conductive portion for heating the volatile liquid, so as to release the odor of the volatile liquid via the capillary structure. Accordingly, the odor generating device can achieve the objective of releasing the odor in accordance with the atmosphere requirement.

### CROSS-REFERENCE TO RELATED APPLICATION

This application is a utility application that claims priority to Taiwan Patent Application entitled, “Odor Generating Device”, having Serial Number 098113477, filed Apr. 23, 2009, which is entirely incorporated herein by reference.

### BACKGROUND OF THE INVENTION

1. Field of the Invention

The invention is related to an odor generating device. More particularly, the invention is related to an odor generating device which is capable of generating a corresponding odor according to an atmosphere requirement.

2. Description of the Prior Art

Nowadays, electronic communication technology is developing rapidly. The different media such as Internet, radio broadcast, and cable TV have already been generalized. Most recently, the development of the third generation mobile communication technology allows people all around the world to communicate with each other via picture, sound and even video. Communication between people has become more comprehensive, by speech and by other's facial and body expressions to fulfill user's sense of sight and hearing.

The creation of a variety of situations has been generally applied in the field of medical science, communication, gaming experience, business conferences, and advertisement marketing. Virtual reality hardware and software are the most popular Some other examples include using a computer screen, a special display equipment or 3D display devices to gain ornamental experience, by applying HI-FI or headphones to gain more sound effects. Some simulation devices can even contain other conceptual processes, for example, some of the advanced systems contain the physical sensation message, which is called a force feedback system.

To provide more realistic sensation experience, in a perfect atmosphere simulation, odor should also be included to fulfill the requirement of the smelling sense or the fifth sense to reach the requirement of simulating an atmosphere.

Since, there is no relative device in prior art capable of generating the odor in accordance with the atmosphere requirement. Accordingly, the prior art fails to provide the interactive and feeling of sense of smell to the user by various communication or multimedia.

With the apparatus of the present invention, an odor can be generated corresponding to the situation to fulfill said demand of the consumer.

### SUMMARY OF THE INVENTION

An objective of the present invention is to provide an odor generating device which is capable of generating an odor according to an atmosphere requirement or a simulated atmosphere. The said odor generating device comprises a conductive portion and a controlling module. The controlling module is connected to the conductive portion for applying a voltage, according to the atmosphere requirement, to the conductive portion for heating the volatile liquid in order to release the odor of the volatile liquid via the capillary structure.

The conductive portion has a capillary structure and a controlling module. The conductive portion is accommodating a volatile liquid having the odor. The controlling module is utilized for releasing the odor of the volatile liquid via the capillary structure, wherein the conductive portion is made of metal and comprises a releasing end and the odor of the volatile liquid is released via the releasing end.

The conductive portion comprises an electrode electrically connected to the controlling module, and the controlling module is used to apply the voltage to the electrode for heating the volatile liquid.

Furthermore, in practice, the odor generating device further comprises a storage unit connected with the conductive portion. The storage unit matches the capillary structure and is used to store the volatile liquid. And the capillary structure of the conductive portion is formed by a metal sintering process or a nano-scale punching process.

Furthermore, the odor generating device is used with a portable multimedia player, wherein the atmosphere requirement is related to contents of a video or audio file.

Another objective of the present invention is to provide an odor generating device which can generate an odor according to an atmosphere requirement. The said odor generating device comprises a conductive portion and a controlling module.

The controlling module is connected to the conductive portion for applying a voltage , according to the atmosphere requirement, on the conductive portion for heating the volatile liquid in order to release the odor of the volatile liquid via the capillary structure.

wherein the conductive portion comprises a releasing end, the odor generating device further comprises a gate disposed at the releasing end of the conductive portion, and the controlling module is electrically connected to the gate for switching between opening and closing the gate according to the atmosphere requirement in order to release the odor of the volatile liquid via the capillary structure and the releasing end.

The conductive portion has a capillary structure for accommodating a volatile liquid having the odor, and a controlling module for releasing the odor of the volatile liquid via the capillary structure, wherein the conductive portion is made of metal and comprises a releasing end. The odor of the volatile liquid is released via the releasing end.

The conductive portion comprises an electrode electrically connected to the controlling module, and the controlling module is used to apply the voltage to the electrode for heating the volatile liquid.

Furthermore, in practice, the odor generating device further comprises a storage unit connected with the conductive portion, the storage unit matches the capillary structure and can be used to store the volatile liquid. And the capillary structure of the conductive portion is formed by a metal sintering process or a nano-scale punching process.

Furthermore, the odor generating device is used with a portable multimedia player, wherein the atmosphere requirement is related to contents of a video or audio file.

Furthermore, the conductive portion is disposed adjacent to or contact with a heat source to heat the volatile liquid, wherein the controlling module switch the state of the gate between opening and closing via a microelectromechanical or nanoelectromechanical system.

The objective of the present invention will no doubt become obvious to those of ordinary skill in the art after reading the following detailed description of the preferred embodiment, which is illustrated in following figures.

### BRIEF DESCRIPTION OF THE APPENDED DRAWINGS

FIG. 1 illustrates a 3D diagram of an appearance of a handheld electronic device 1.

FIG. 2 illustrates a function block diagram of the odor generating device according to FIG. 1.

FIG. 3 illustrates a function block diagram of the odor generating device according to another embodiment of the invention.

FIG. 4 illustrates a 3D diagram of an appearance of a handheld electronic device 1 with an odor generating device according to another embodiment of the invention.

FIG. 5 illustrates a function block diagram of the odor generating device according to FIG. 4.

FIG. 6 illustrates a function block diagram of the odor generating device according to another embodiment of the invention.

### DETAILED DESCRIPTION OF THE INVENTION

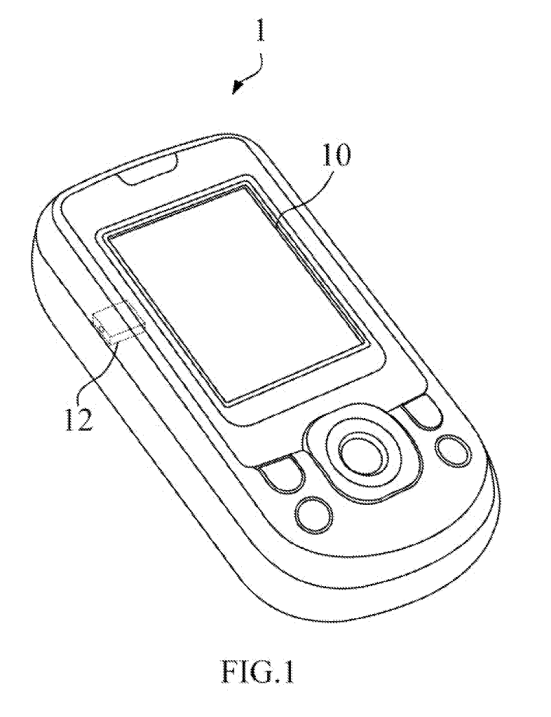

Please refer to FIG. 1. FIG. 1 illustrates a 3D diagram of an appearance of a handheld electronic device 1, the handheld electronic device 1 comprises an odor generating device 12 according to an embodiment of the invention. As shown in FIG. 1, the handheld electronic device 1 comprises a playing module 10 and an odor generating device 12.

The odor generating device 12 is capable of performing on the handheld electricity module 10. In this embodiment, the odor generating device 12 is disposed in the handheld electronic device 1. In practice, the odor generating device 12 can, but not limited to, be used independently.

The handheld electronic device 1 can be a portable multimedia playing device which is capable of playing an audio-visual file, for example, a personal digital assistant, a smart-phone, a portable media player and other devices which are capable of playing the audio-visual file. As shown in FIG. 1, the handheld electronic device 1 can be, but not limit to, a smart-phone which is capable of playing the audio-visual file.

While the playing module 10 of handheld electronic device 1 is utilized to play the audio-visual file, the contents of the audio-visual file are related to a relative atmosphere requirement.

The odor is generated in accordance with an atmosphere requirement or a simulated atmosphere. The simulated atmosphere is simulated in accordance with a multimedia. In practice, the multimedia may pertain to an audio file, the audio-visual file or an audio-visual stream.

For example, while the atmosphere is about a garden, the odor generating device 12 generates or releases the odor of potpourri in accordance with the atmosphere requirement.

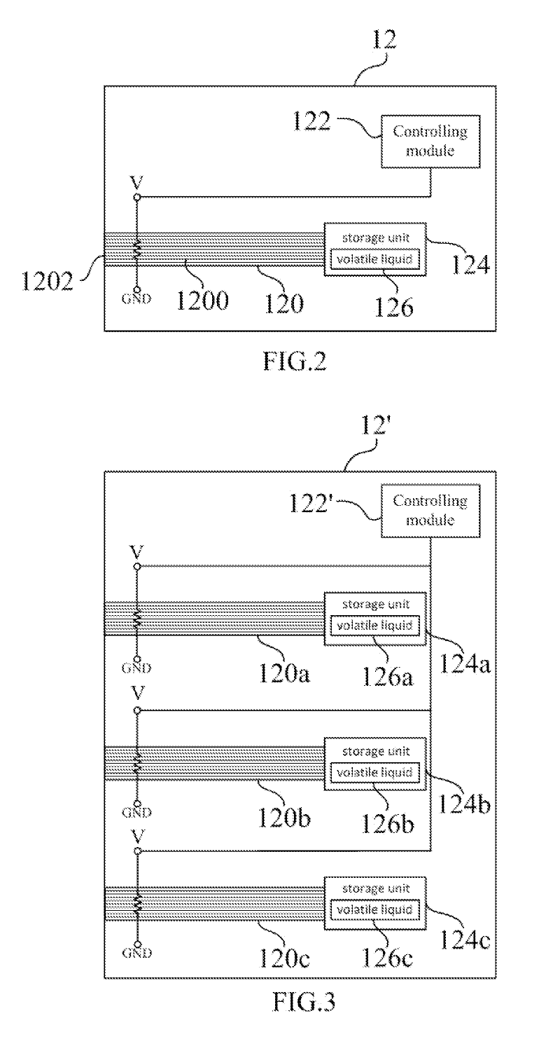

Please refer to FIG. 2. FIG. 2 illustrates a function block diagram of the odor generating device according to FIG. 1. As shown in FIG. 2, the odor generating device 12 comprises a conductive portion 120, a controlling module 122 and a storage unit 124.

The conductive portion 120 can be formed by porous metal with the capillary structure 1200. To form the capillary structure 1200 of conductive potion 120, large amount of tiny pores can be formed by metal sintering or nanometer class punching process. By the principle of capillarity, the capillary structure 1200 can be utilized to accommodate the volatile liquid 126 and to allow the transmission of volatile liquid 126.

The conductive portion 120 is disposed in the odor generating device 12. Furthermore, the conductive portion 120 has a releasing end 1202, the releasing end 1202 is mounted on the surface of the odor generating device 12 and exposed to the surface thereof, which means the conductive portion 120 is connected to the outer space via the releasing end 1202.

The conductive portion 120 has an electrode V and a corresponding end of the ground GND. The controlling module 122 is electrically connected to conductive portion 120 by electrode V. Since the conductive portion 120 has the characteristics of metal, the resistance may be the equivalent form resistive heater by applying a voltage on the resistance formed of the conductive portion 120.

The controlling module 122 heat the capillary structure 1200 by applying the voltage thereon to gasify the volatile liquid 126. The gasified volatile liquid 126 in accordance with the atmosphere requirement passes through the releasing end 1202 of the conductive portion 120 and is released to the air.

In an embodiment, by tuning the controlling module 122, the voltage can be controlled. Accordingly, the temperature can also be adjusted to control the concentration and the releasing rate of the odor.

After releasing of volatile liquid 126 via the releasing end 1202 of conductive portion 120, the volatile liquid 126 in the capillary structure 1200 of the outer end (releasing end 1202) of the conductive portion 120 reduces due to the gasification.

By the capillary phenomenon and the natural phenomena of high concentration to low concentration, the reduction of the volatile liquid 126 of the outer end of the conductive portion 120 may draw the volatile liquid 126 out from the inner side to the outside of the conductive portion 120, in order to transmit the volatile liquid 126 via the capillary structure 1200 of the conductive portion 120.

In another word, the capillary structure 1200 of conductive portion 120 can be utilized as a transmission path and a housing for accommodating the liquid with odor.

Additionally, as shown in FIG. 2, the storage unit 124 of the odor generating device 12 is connected with the conductive portion 120. The storage unit 124 corresponds to the capillary structure 1200 and can be used to support accommodation of the volatile liquid 126. Furthermore, the storage unit improves the persistence of the odor generated by the odor generating device 12. Additionally, the storage unit can be a refillable housing or a replaceable container for allowing user to refill, replace, and survey the state of the volatile liquid 126 conveniently.

In another embodiment, in order to generate the odor in accordance with the atmosphere requirement, the amount of conductor 120 installed in the odor generating device 12 with the capillary structure 1200 is not limited to one.

The conductive portion 120 may contains a plurality of the conductive portion 120 which contains various volatile liquids 126. Furthermore, more diverse odor for simulating the atmosphere can be produced by mixing more than one odors together with certain ratios.

Please refer to FIG. 3. FIG. 3 illustrates a function block diagram of the odor generating device 12′ according to another embodiment of the invention. In comparison with the previous embodiments, the odor generating device 12′ comprises a conductive portion 120a, a conductive portion 120b and a conductive portion 120c.

The conductive portion 120a, the conductive portion 120b and the conductive portion 120c comprise a capillary structure 1200 respectively for accommodating various volatile liquids 126a, 126b, 126c. Each of the capillary structures of the three conductive portions 120a, 120b, 120c is able to match the storage unit 124a, 124b, 124c correspondingly.

Accordingly, at least three different odors can be released. The other structures of the odor generating device 12′ are approximately the same as the descriptions mentioned above and the discussion of unnecessary details will be hereby omitted.

In further embodiment, the controlling module 122′ of the odor generating device 12′ is capable of applying a voltage on different conductive portions to release more than one odor at the same time to mix the odor in a different ratios or concentrations.

For example, odor generating device 12′ releases a basic odor containing 30% of coconut oil and another basic odor containing 70% of sea salt. The odor for simulating the atmosphere corresponding to the atmosphere requirement of ocean is formed by mixing the said odors together. Accordingly, the odor generating device 12′ supports more diverse effects of odor for simulating the atmosphere.

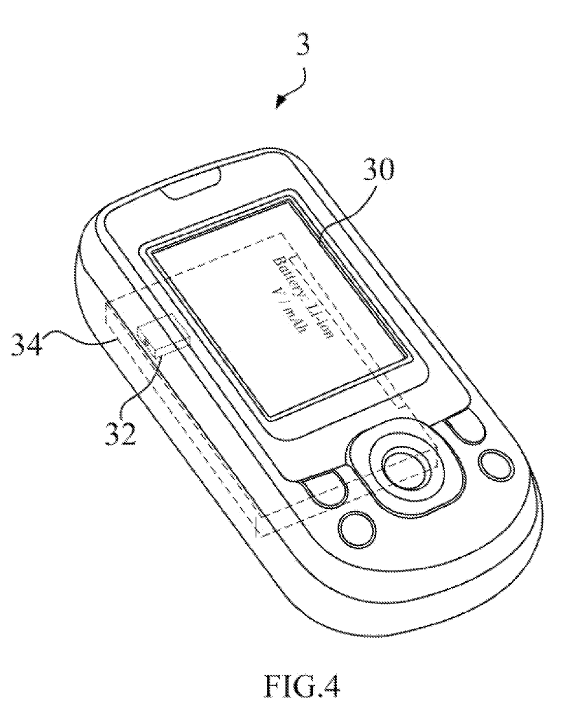

Please refer to FIG. 4. FIG. 4 illustrates a 3D diagram of an appearance of a handheld electronic device 3 with an odor generating device 32 according to another embodiment of the invention. As shown in FIG. 4, the handheld electronic device 3 comprises a playing module 30, an odor generating device 32 and a heat source 34.

In this embodiment, the handheld electronic device 3 can be a portable multimedia playing device, the heat source 34 of the handheld electronic device 3 can be a battery module of the handheld electronic device 3. In practice, several units or modules in the common handheld electronic device 3 are relatively easy to generate heat in the progresses of computing or supplying electricity, for example, cell modules, processing modules and transformer modules which the energy waste is not avoidable.

The odor can be released by exploiting the heat source 34 of the odor generating device 32 as the heating module to save power and to maximize the use of the results.

Please also refer FIG. 5. FIG. 5 illustrates a function block diagram of the odor generating device according to FIG. 4. As shown in FIG. 5, the odor generating device 32 comprises a conductive portion 32, a gate 321, a controlling module 322 and a storage unit 324.

The conductive portion 320 can be formed by porous metal with capillary structures 3200. The capillary structure 3200 of the conductive portion 320 is formed by a metal sintering process or a nano-scale punching process.

By the principle of capillarity, the capillary structure 3200 can be utilized to accommodate the volatile liquid 326, which also allows the transmission of volatile liquid 326.

As shown in FIG. 5, the conductive portion 320 is disposed in the odor generating device 32. Furthermore, the conductive portion 320 has a releasing end 3202. The releasing end 3202 is exposed to the surface of the odor generating device 12, which means conductive portion 320 is linked to the outer space via the releasing end 3202.

Additionally, the odor generating device 32 and the conductive portion 32 of the odor generating device 32 are disposed close to or in contact with the heat source 34 of the handheld electronic device. Thus, the volatile liquid 326 stored in the conductive portion 320 can be heated directly or indirectly by the heat source.

Gate 321 is disposed at the releasing end of the conductive portion 320. The gate 321 is switchable. The gate 321 can be controlled by a micro-electromechanical system. The opening or closing of the gate 321 is controlled by the micro-electromechanical system. The control module 322 is electronically connected with the gate 321. The control module 322 controls the gate 321 via the micro-electromechanical system.

When the atmosphere requirement has not yet created by the handheld electronic device and the odor generating device 32 has not receive the atmosphere requirement.

The gate 321 is at a closing state. The volatile liquid 326 accommodated in the capillary structure 3200 of conductive portion 320 is heated and be gasified by the heat source 34. However, the gate 321 is in closing state, so the high pressure gas generated by volatile liquid 326 is enclosed in the capillary structure 3200 of conductive portion 320.

While an atmosphere requirement is created by the handheld electronic device 1, for example, the audio-visual file is playing by the playing module 30 and a corresponding odor is required, the controlling module 322 opens the gate 321 via micro-electromechanical system while an atmosphere requirement is received by the odor generating device 32. Meanwhile, the high pressure gas with the corresponding odor is generated by pre-heating the volatile liquid 326, said high pressure gases will be released via the releasing end 3202 of the conductive portion 320 subsequently.

Hereafter, the volatile liquid 326 accommodated in the capillary structure 3200 corresponding to the atmosphere requirement keeps releasing odor during the switchable gate is at opening state. When the atmosphere requirement ends, the controlling module 322 closes the gate via micro-electromechanical system.

In this embodiment, capillary structure 3200 can be treated as a transmitting media of the volatile liquid 326 and also be the housing for accommodating the high pressure volatile liquid 326. The odor generating device 32 can control the gate 321 disposed at the releasing end 3202 to control the releasing rate of the odor according to the atmosphere requirement.

In an embodiment, by tuning the opening range or the opening time of the gate 321, the releasing rate and concentration of odor can both be adjusted

After the volatile liquid 326 passed through the releasing end of conductive portion 320 and the odor is released, the volatile liquid 326 in the capillary structure 3200 of the releasing end of the conductive portion 320 reduces due to the gasification of the volatile liquid 326. As the capillary phenomenon and the natural phenomena of high concentration to low concentration, the capillary structure of conductive portion can be utilized as the transmission path and housing for accommodating the odor liquid.

Furthermore, as shown in FIG. 5, the storage unit 324 of the odor generating device 32 is connected with the conductive portion 320. The storage unit 324 is corresponding to the capillary structure 3200 and capable of supporting the accommodation of volatile liquid 326. The storage unit 324 is capable of improving the persistence of the odor generated by the odor generating device 32. Additionally, for allowing users to conveniently refill, replace, and survey the state of the volatile liquid 326, the storage unit 324 can be a refillable housing or a replaceable package.

In another embodiment, the amount of conductor 320 of odor generating device 32 with the capillary structure 3200 is not limited to one, the conductive portion 320 may contains a plurality groups of the conductive portion 320 with varies volatile liquid 326. Furthermore, by mixing more than one odors together in certain ratios, more diverse odor for simulating the atmospheres can be produced.

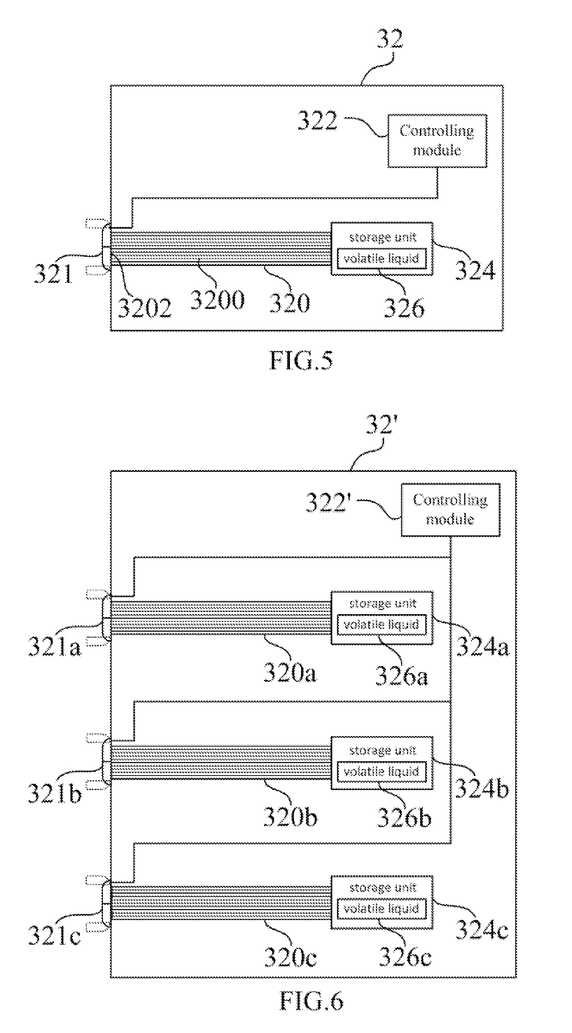

Please refer to FIG. 6. FIG. 6 illustrates a function block diagram of the odor generating device 32′ according to another embodiment of the invention. In comparison with the prior art, the odor generating device 32′ comprises a conductive portion 320a, a conductive portion 320b and a conductive portion 320c. Furthermore, the odor generating device 32′ has the gate 321a, gate 321b and gate 321c corresponding to the three conductive portions 320a, 320b, 320c respectively.

The conductive portion 120a, conductive portion 120b and 120c respectively having the corresponding capillary structure, the controlling module 322′ of odor generating device 32′ is electrically connected with the three gates of the conductive portion which correspond to the three conductive portions.

The controlling module 322′ of the odor generating device 32′ is electrically connected with the said three gates, and to control the state of the gates via the micro-electromechanical system. The switch mechanism, as mentioned above and discussion of unnecessary detail will be hereby omitted. Accordingly, the odor generating device 32′ is capable of releasing varies odors according to the atmosphere requirement.

In the further embodiment, by opening more than one gates simultaneously by controlling the controlling module 322′ of the odor generating device 32′, more than one odors are mixed together with certain ratios, more diverse odor for simulating the atmospheres can be produced.

The present invention is to provide an odor generating device, which is capable of releasing an odor according to an atmosphere requirement, atmosphere requirement to form an electronic device which allows the user to sense the odor corresponding to the atmosphere requirement.

Furthermore, the capillary structure of present invention is formed by metal sintering or nanometer class punching process. The capillary structure having the characteristic of light weight and small size, which can also be utilized to be a housing for accommodating or transmitting tunnel of the volatile liquid, or even be utilized as a portion of heating mechanism for heating the volatile liquid. By matching the chip class controlling module, the odor generating device of present invention fulfills the requirement of small size, high mobility and low electricity consumption, which can be widely utilized in varies portable electronic device.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. An odor generating device for generating an odor according to an atmosphere requirement, comprising:
a conductive portion having a capillary structure for accommodating a volatile liquid having the odor; and
a controlling module for releasing the odor of the volatile liquid via the capillary structure.

2. The odor generating device of claim 1,
wherein the controlling module is electrically connected to the conductive portion, and used to apply a voltage, according to the atmosphere requirement, to the conductive portion for heating the volatile liquid in order to release the odor of the volatile liquid via the capillary structure.

3. The odor generating device of claim 2, wherein the conductive portion comprises an electrode, electrically connected to the controlling module, and the controlling module is used to apply the voltage to the electrode for heating the volatile liquid.

4. The odor generating device of claim 1, further comprising:
a storage unit connected with the conductive portion, the storage unit matching the capillary structure and used to store the volatile liquid.

5. The odor generating device of claim 1, wherein the capillary structure of the conductive portion is formed by a metal sintering process or a nano-scale punching process.

6. The odor generating device of claim 1 used with a portable multimedia player.

7. The odor generating device of claim 6, wherein the atmosphere requirement is related to contents of a video or audio file.

8. The odor generating device of claim 1, wherein the conductive portion is made of metal.

9. The odor generating device of claim 1, wherein the conductive portion comprises a releasing end and the odor of the volatile liquid is released via the releasing end.

10. The odor generating device of claim 1, wherein the conductive portion comprises a releasing end, the odor generating device further comprises a gate disposed at the releasing end of the conductive portion, and the controlling module is electrically connected to the gate for switching between opening and closing the gate according to the atmosphere requirement in order to release the odor of the volatile liquid via the capillary structure and the releasing end.

11. The odor generating device of claim 10,
wherein the controlling module is electrically connected to the conductive portion, and used to apply a voltage, according to the atmosphere requirement, to the conductive portion for heating the volatile liquid in order to release the odor of the volatile liquid via the capillary structure.

12. The odor generating device of claim 11, wherein the conductive portion comprises an electrode, electrically connected to the controlling module, and the controlling module is used to apply the voltage to the electrode for heating the volatile liquid.

13. The odor generating device of claim 10, a storage unit connected with the conductive portion, the storage unit matching the capillary structure and used to store the volatile liquid.

14. The odor generating device of claim 10, wherein the capillary structure of the conductive portion is formed by a metal sintering process or a nano-scale punching process.

15. The odor generating device of claim 10 used with a portable multimedia player.

16. The odor generating device of claim 15, wherein the atmosphere requirement is related to contents of a video or audio file.

17. The odor generating device of claim 10, wherein the conductive portion is made of metal.

18. The odor generating device of claim 10, wherein the conductive portion comprises a releasing end and the odor of the volatile liquid is released via the releasing end.

19. The odor generating device of claim 10, wherein the conductive portion is disposed adjacent to or in contact with a heat source to heat the volatile liquid.

20. The odor generating device of claim 10, wherein the controlling module switching between opening and closing the gate via a microelectromechanical or nanoelectromechanical system.
